(19) United States
(12) Patent Application Publication
(10) Pub. No.: US 2019/0231801 A1
(43) Pub. Date: Aug. 1, 2019

(54) METHODS OF TREATING OVARIAN CANCER

(71) Applicant: Cipla Limited, Mumbai (IN)

(72) Inventors: Geena Malhotra, Mumbai (IN); Kalpana Joshi, Maharashtra (IN)

(21) Appl. No.: 16/148,014

(22) Filed: Oct. 1, 2018

Related U.S. Application Data
(63) Continuation of application No. 15/381,221, filed on Dec. 16, 2016, now abandoned.

(30) Foreign Application Priority Data
Dec. 16, 2015 (IN) 4715/MUM/2015

(51) Int. Cl. A61K 31/65; A61K 9/00; A61K 47/02; A61K 31/337; A61K 33/24; A61K 45/06; A61K 9/48; A61K 9/20; A61K 9/08; A61K 47/26; A61K 47/20; A61K 47/18; A61K 47/14; A61K 47/12; A61K 47/10; A61K 31/138

(52) U.S. Cl. CPC A61K 31/65; A61K 31/138; A61K 47/02; A61K 31/337; A61K 33/24; A61K 45/06; A61K 9/4866; A61K 9/485; A61K 9/2059; A61K 9/2054; A61K 9/2018; A61K 9/2009; A61K 9/08; A61K 47/26; A61K 9/0095; A61K 47/20; A61K 47/183; A61K 47/18; A61K 47/14; A61K 47/12; A61K 47/10; A61K 9/0019

(57) ABSTRACT

Disclosed herein are methods of treating ovarian cancer comprising administering to a subject in need thereof an effective amount of methacycline, or a pharmaceutically acceptable salt thereof. The methacycline can be administered as part of treatment regimen, which also can include other chemo- or immune-therapies, radiation therapy, and/or surgical treatments.

### CROSS REFERENCE TO RELATED APPLICATION

This application claims the benefit of Indian Application 4715/MUM/2015, filed Dec. 16, 2015, the contents of which are hereby incorporated by reference in its entirety.

### FIELD OF THE INVENTION

The invention relates to methods for the treatment of ovarian cancer using tetracycline-type antibiotics, in particular methacycline. The invention also relates to pharmaceutical compositions containing tetracycline antibiotics such as methacycline that are useful for the treatment of ovarian cancer.

### BACKGROUND

Ovarian cancer is the fifth leading cause of cancer death in women today according to the National Cancer Institute (NCI) 2014 statistics. Ovarian cancer is most common in women who have been through the menopause (usually over the age of 50), although it can affect women of any age. It is diagnosed in approximately 22,000 women in the United States of America yearly and accounts for at least 14,000 deaths. Approximately two thirds of women are diagnosed with stage III or IV disease, stages characterized by a five-year survival of 25-30% or less. In comparison, the five-year survival for those with stage I or II disease is 85-90%.

The ovaries are the main source of female hormones estrogen and progesterone. Ovarian cancer often starts silently, meaning symptoms are not detectable until its later stages. Ovarian cancer can spread by local extension, lymphatic invasion, intraperitoneal implantation, hematogenous dissemination, and transdiaphragmatic passage. Intraperitoneal dissemination is the most common and recognized characteristic of ovarian cancer.

The five major histological subtypes of ovarian cancer have unique genomic, epidemiological, and histological features and are now viewed as distinct malignancies. The treatment of ovarian cancer is based on the type of the cancer, stage and grade of the disease. Treatment includes surgery, chemotherapy, radiotherapy, hormonal therapy and targeted therapy. Epithelial tumors account for about 90% of ovarian cancers, and are the leading cause of death from gynecological malignancies whereas sex cord stromal and germ cell tumors account for the remaining ˜10% of ovarian cancers. Most common gene mutations in ovarian cancer occur in NF1, BRCA1, BRCA2, 125/CA125 and CDK12. Type-I ovarian cancers tend to have microsatellite instability in several genes, including BRAF, KRAS, and PTEN, which are tumor suppressor genes. Type-II cancers have different genes mutated, including p53, BRCA1, and BRCA2.

Currently FDA approved drugs for the treatment of ovarian cancer include bevacizumab, pazopanib, cyclophosphamide, doxorubicin, gemcitabine hydrochloride, topotecan hydrochloride, olaparib, carboplatin, cisplatin, paclitaxel, thiotepa and combinations such as bleomycin+etoposide+cisplatin, carboplatin+taxol, carboplatin+gemcitabine, carboplatin+pegylated liposomal doxorubicin, gemcitabine+cisplatin, vinblastine sulfate+ifosfamide+cisplatin. While the aforementioned drugs can treat ovarian cancer with varying levels of success, they are usually accompanied with severe negative side effects.

There remains a need for effective, non-surgical treatments of ovarian cancer. There remains a need for agents effective to treat ovarian cancer with reduced side effect profiles relative to currently used medications.

### SUMMARY

Disclosed herein are methods of treating ovarian cancer. The methods include administering to a patient in need thereof methacycline in an amount effective to treat the ovarian cancer. In some instances, methacycline can be administered as part of a combination therapy. Also disclosed herein are pharmaceutical compositions containing methacycline suitable for the treatment of ovarian cancer. In some instances, the compositions include an additional anti-cancer agent.

The details of one or more embodiments are set forth in the descriptions below. Other features, objects, and advantages will be apparent from the description and from the claims.

### BRIEF DESCRIPTION OF THE FIGURES

FIG. 1 includes a depiction of the absolute IC₅₀ values (μM) for methacycline against human ovarian cancer cell lines.

FIG. 2 includes a depiction of a concentration response curve for methacycline against human ovarian cancer cell lines.

FIG. 3 includes a depiction of a concentration response curve for paclitaxel against human ovarian cancer cell lines.

FIG. 4 includes a depiction of the absolute IC₅₀ values (μM) for paclitaxel against human ovarian cancer cell lines.

FIG. 5 includes a depiction of the efficacy of methacycline against human ovarian tumor models.

FIG. 6 includes a depiction of the efficacy of paclitaxel against human ovarian tumor models.

FIG. 7 includes a depiction of the anti-tumor efficacy of Methacycline in combination with paclitaxel in OVXF SK-OV-3. Left: Modeled T/C, which is the mean of experimental T/C for each pair of conditions in the combination matrix. Right: Bliss index, which is the difference of Bliss neutral and modeled T/C for each pair of conditions.

FIG. 8 includes a depiction of the anti-tumor efficacy of methacycline in combination with paclitaxel in OVXF A2780. Left: Modeled T/C, which is the mean of experimental T/C for each pair of conditions in the combination matrix. Right: Bliss index, which is the difference of Bliss neutral and modelled T/C for each pair of conditions.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Methacycline is a tetracycline antibiotic, effective against Mycobacterium avium complex (MAC) and has been used for the treatment of acute bacterial exacerbations of chronic bronchitis. It has also used to treat respiratory tract infections, sexually transmitted infections, otitis media, and AIDS-related infections. Methacycline binds to the 16S part of the 30S ribosomal subunit and prevents the amino-acyl tRNA from binding to the A site of the ribosome. Methacycline has the following chemical structure:

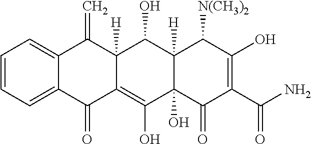 [SMILES: C=C1c2ccccc2C(=O)C2=C(O)[C@]3(O)C(=O)C(C(N)=O)=C(O)[C@@H](N(C)C)[C@@H]3[C@@H](O)[C@H]12]

The present inventors have found that methacycline exhibits a significant role in the treatment of ovarian cancer. It has been found that methacycline has a therapeutic effect on matrix metalloproteinases (MMPs) which are frequently expressed in ovarian cancer and plays an important role in the metastatic process. MMPs are considered as potential diagnostic and prognostic biomarkers in many types and stages of cancer. MMPs may contribute to the antiapoptotic effect by activating indirectly the serine/threonine kinase Akt/protein kinase B through the signaling cascades of EGFR and IGFR. MMP overexpression is associated with an increased metastatic potential of ovarian tumors, which leads to poor prognosis and decreased survival. In the tumor microenvironment, MMPs may be the key regulatory point in disrupting the balance between growth and antigrowth signals thereby influencing the bioavailability of growth factors to stimulate tumor cell growth.

Disclosed herein are methods of treating ovarian cancer in a patient in need thereof by administering an effective amount of methacycline. Unless stated to the contrary, the term methacycline refers both to methacycline free base and pharmaceutically acceptable salts thereof.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, ptoluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made. Pharmaceutically acceptable anions include the conjugate bases of the acids listed above. Preferred salts forms include methacycline hydrochloride and methacycline sulfate.

Preferably, methacycline may be administered to the subject once daily, twice daily or thrice daily. A typical recommended daily dosage regimen can range from about 5 mg to 5,000 mg, from about 10 mg to 4,000 mg, from about 100 mg to 4,000 mg, from about 500 mg to 4,000 mg, from about 500 to 2,000 mg, from about 1,000 to 2,000 mg, from about 1,000 to 3,000 mg, from about 1,500 to 2,500 mg, from about 500 to 1,500 mg, or from about 2,000 to 4,000 mg. More preferably, methacycline may be administered to the subject once daily, twice daily or thrice daily in a typical dosage regimen that can range from 1,000 to 1,500 mg.

Preferably, the active agent may be provided in the form of a pharmaceutical composition such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution and sprinkles, transdermal patches, however, other dosage forms such as controlled release formulations, lyophilized formulations, modified release formulations, delayed release formulations, extended release formulations, pulsatile release formulations, dual release formulations and the like. Liquid and semisolid dosage forms (liquids, suspensions, solutions, dispersions, ointments, creams, emulsions, microemulsions, sprays, patches, spot-on), parenteral, topical, inhalation, buccal, nasal etc. may also be envisaged under the ambit of the invention. The inventors of the present invention have also found that the solubility properties of the active agent may be improved by nanosizing thus leading to better bioavailability and dose reduction of the drug.

In one embodiment, methacycline may be present in the form of nanoparticles which have an average particle size of less than 2,000 nm, less than 1,500 nm, less than 1,000 nm, less than 750 nm, less than 500 nm, or less than 250 nm.

Suitable excipients may be used for formulating the dosage form according to the present invention such as, but not limited to, surface stabilizers or surfactants, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof.

In some embodiments, methacycline can be administered in an amount effective to inhibit the activity of matrix metalloproteinases in ovarian cancer cells. In some instances, methacycline can be administered in an amount effective to inhibit matrix metalloproteinase-2 and/or matrix metalloproteinase-9 in ovarian cancer cells. In some embodiments, methacycline can be administered in an effective amount to treat ovarian cancers. For instance, methacycline can reduce tumor size, inhibit tumor growth, alleviate symptoms, delay progression, prolong survival, including, but not limited to disease free survival, prevent or delay ovarian cancer metastasis, reduce or eliminate preexisting ovarian cancer metastasis, and/or prevent recurrence of ovarian cancer.

As used herein, the terms “reduce” or “delay” refers to methods that reduce the probability of disease development/extent in a given time frame, when compared to otherwise similar methods that do not include the use of methacyline. Probabilities can be established using clinical trials, but can also be determined using in vitro assays when correlations have been established. In some embodiments, methacycline can inhibit ovarian cancer cell proliferation. For instance, at least about 10%, 20%, 30%, 40%, 60%, 70%, 80%, 90%, or 100% of cell proliferation is inhibited. In some embodiments, methacycline can inhibit ovarian cancer metastasis. For instance, at least about 10%, 20%, 30%, 40%, 60%, 70%, 80%, 90%, or 100% of metastasis is inhibited.

Ovarian cancer can be characterized by overall stage, 0-IV, as well as using the FIGO system. Methacycline may be administered to patients at various stages of ovarian cancer. For instance, methacycline may be administered to a patient at Stage 1A (T1a, N0, or M0), Stage IB (T1b, N0, M0), Stage IC (T1c, N0, M0), Stage IIA (T2a, N0, M0), Stage IIB (T2b, N0, M0), Stage IIIA1 (T1 or T2, N1, M0), Stage IIIA2 (T3a2, N0 or N1, M0), Stage IIIB (T3b, N0 or N1, M0), Stage IIIC (T3c, N0 or N1, M0), Stage IVA and/or Stage IVB. Ovarian cancer can include fallopian tube cancer, as well as primary peritoneal cancer (i.e., cancer around but not within the ovaries or fallopian tubes. In certain embodiments, ovarian cancer refers to cancer inside the ovaries and/or fallopian tubes. Methacycline can be used to treat epithelial stromal tumors, for instance serous, mucinous, endometrioid, clear cell, or brenner type tumors.

Methacycline may be used for the treatment of ovarian cancer in mammals, especially humans, in monotherapy mode or in a combination therapy (e.g., dual combination, triple combination etc.) mode such as, for example, in combination with one or more anti-cancer therapeutics.

Methacycline can be administered to ovarian cancer patients also receiving one or more immunotherapeutic agents. Immunotherapies include monoclonal antibody, i.e., checkpoint inhibitors, and oncolytic virus. Oncolytic viruses are genetically engineered or naturally occurring viruses that selectively replicate in and kill cancer cells without harming the normal tissues. The viruses are modified such that they can replicate in cancerous cells, but not healthy cells.

In certain embodiments, especially those involving stromal tumors, methacycline can be administered in conjunction with one or more hormone therapy agents. For instance, methacycline can be administered with a tamoxifen, an aromatase inhibitor (e.g., letrozole, anastrozole or exemanstane), or a lutenizing hormone releasing hormone (LHRH) agonist (goserelin, leuprolide).

In cases of combination therapy, it is possible that a unitary dosage form containing both methacycline and additional anti-cancer agent may be employed. In some instances, the combinations may be provided in form suitable for parenteral application such as but not limited to injection. In some instances, methacycline is administered in a separate dosage form from any other additional anti-cancer agent.

In some embodiments, methacycline can be administered as part of a surgical or radiological treatment regime. For instance, a patient may be administered methacycline prior to and/or after undergoing hysterectomy (for instance, a total hysterectomy) and/or bilateral salpingo-oophorectomy. In some instances, methacycline can be administered as part of a debulking, or intervallic debulking treatment regimen. Likewise, a patient may be administered methacycline prior to and/or after undergoing radiation therapy.

Methacycline can be administered as part of a treatment regime that includes surgical and chemotherapeutic components. The patient, in addition to receiving one or more of the anti-cancer agents identified above, can receive methacycline prior to and/or after undergoing a surgical procedure. In some embodiments, methacycline can be administered as part of a treatment regime that includes radiation therapy and chemotherapeutic components. The patient, in addition to receiving one or more of the anti-cancer agents identified above, can receive methacycline prior to and/or after undergoing radiation therapy.

Methacycline can be used in combination with one or more anti-cancer agents. The term “anti-cancer drug” is used in broad sense to include, but is not limited to, oncolytic viruses, monoclonal antibodies, microtubule inhibitors, topoisomerase inhibitors, platins, alkylating agents, and anti-metabolites. Suitable drugs which can be used in combination with methacycline include bevacizumab, bleomycin, ifosfamide, etoposide, doxorubicin (including PEGylated doxorubicin), pazopanib, cyclophosphamide, doxorubicin, gemcitabine, vinblastine, topotecan, olaparib, carboplatin, cisplatin, paclitaxel, and thiotepa. In some instance, methacycline can be administered as part of another combination, for instance, bleomycin+etoposide+cisplatin, carboplatin+taxol, carboplatin+gemcitabine, carboplatin+pegylated liposomal doxorubicin, gemcitabine+cisplatin, vinblastine sulfate+ifosfamide+cisplatin.

In order that this invention be more fully understood, the following preparative and testing methods are set forth. These methods are for the purpose of illustration only and are not to be construed as limiting the scope of the invention in any way.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: In Vitro Cytotoxicity 2D Assay

In the present study the human ovarian cancer cell lines OVXF 899, OVXF 1023, A2780, SK-OV-3, OVCAR-3, IGR-OV1, OVCAR-5 and NCI/ADR-RES were used. OVXF 899 and OVXF 1023 were established at Oncotest, Germany from the corresponding human patient derived xenograft. A2780, IGR-OV1, NCI/ADR-RES, OVCAR-3 and OVCAR-5 were kindly provided by the NCI (National Cancer Institute, Bethesda, Md., USA). SK-OV-3 was purchased from ATCC (Rockville, Md., USA). Authenticity of cell lines was confirmed at the DSMZ by STR (short tandem repeat) analysis, a PCR based DNA-fingerprinting methodology. Cell lines were routinely passaged once or twice weekly and maintained in culture for up to 20 passages. All cells were grown at 37° C. in a humidified atmosphere with 5% CO2 in RPMI 1640 medium (25 mM HEPES, with L-glutamine, #FG1385, Biochrom, Berlin, Germany) supplemented with 10% (v/v) fetal calf serum (Sigma, Taufkirchen, Germany) and 0.1 mg/mL gentamicin (Life Technologies, Karlsruhe, Germany).

The CellTiter-Blue® Cell Viability Assay (#G8081, Promega) was used according to manufacturer's instructions. Briefly, cells were harvested from exponential phase cultures, cells/well depending on the cell line's growth rate. After a 24 h recovery period to allow the cells to resume exponential growth, test compounds were added.

Following an incubation period of up to four hours, fluorescence (FU) was measured by using the Enspire Multimode Plate Reader (excitation k=531 nm, emission k=615 nm). For calculations, the mean values of duplicate/six fold (untreated control) data were used. Sigmoidal concentration-response curves were fitted to the data points (T/C values) obtained for each cell line using 4 parameter non-linear curve fit (Oncotest Warehouse Software).

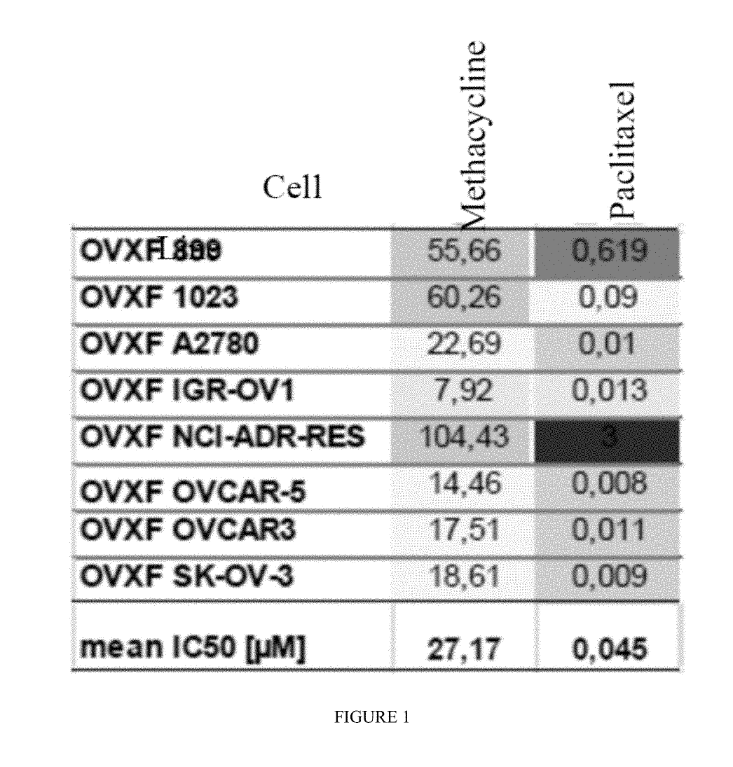

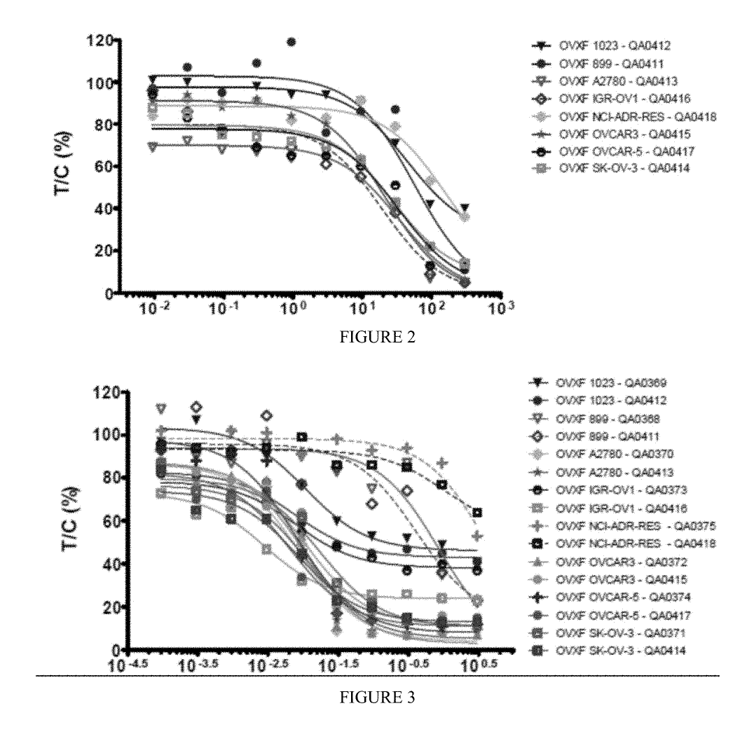

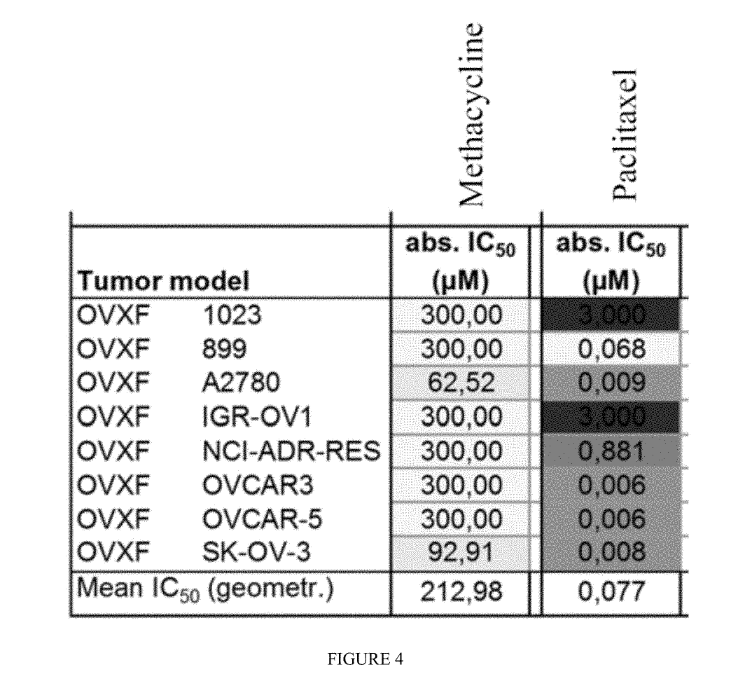

The in vitro anti-tumor activity of methacycline in eight selected human ovarian cancer cell lines were evaluated. The test compounds was applied at 10 concentrations in half-log increments up to 300 μM. As a reference compound paclitaxel was tested in parallel. Cells were treated for 96 h with the test compounds. Anti-tumor activity was assessed by using the CellTiter-Blue® Cell Viability Assay. Potency is expressed as absolute IC₅₀ and relative IC₅₀ values, calculated by non-linear regression analysis. Results are depicted in FIGS. 1-4.

Methacycline showed a geometric mean absolute IC₅₀ value of 27.17 μM. Individual IC₅₀ values were in the range from 7.92 μM (IGR-OV1) to 104.43 μM (NCI/ADR-RES), corresponding to 13-fold difference between the most sensitive and most resistant cell line. Selected activity as defined as individual IC₅₀ values smaller than ½ of mean IC₅₀ value) was evident for the cell line IGR-OV1 (individual IC₅₀=7.92 μM). In addition, above-average activity was found in the cell lines OVCAR-5 (IC₅₀=14.46 μM), OVCAR-3 (IC₅₀=17.51 μM), SK-OV-3 (IC₅₀=18.61 μM) and A2780 (IC₅₀=22.69 μM). The reference compound paclitaxel showed concentration-dependent activity in all cell lines tested with a geometric mean absolute IC₅₀ value of 0.045 μM.

### Example 2: In Vitro Cytotoxicity 3D Assay

In the present study methacycline and the reference compound paclitaxel were investigated for anticancer activity ex vivo in eight human tumor cell lines of ovarian cancer. Tests were carried out using a 3D clonogenic assay in 96-well format with image based read-out. The aim of the study was to investigate antitumor potency and tumor type selectivity of the compound. Cell lines were routinely passaged one or twice weekly. All cells were grown at 37° C. in a humidified atmosphere with 5% CO₂ in RPMI 1640 medium (Biochrom) supplemented with 10% (v/v) fetal calf serum and 0.1 mg/mL gentamicin. The percentage of viable cells was determined in a Neubauer-hemocytometer using trypan blue exclusion.

The clonogenic assay was carried out in a 96 well plate format using ultra low attachment plates. For each test, cells were prepared as described above and assay plates were prepared as follows: each test well contained a layer of semi-solid medium with tumor cells (50 and a second layer of medium supernatant with or without test compound (100 The cell layer consisted of 2·10³ to 3·10³ tumor cells per well, which were seeded in 50 μL/well cell culture medium (IMDM, supplemented with 20% (v/v) fetal calf serum, 0.01% (w/v) gentamicin, and 0.4% (w/v) agar. After 24 hours the test compounds were added after serial dilution in cell culture medium, and left on the cells for the duration of the experiment (continuous exposure, 100 μl drug overlay). Test concentrations ranged from 0.01 μM to 316.2 μM (methacycline), and 0.000095 μM to 3 μM (paclitaxel). Every plate included six untreated control wells and drug-treated groups in duplicate at 9 concentrations. Cultures were incubated at 37° C. and 7.5% CO₂ in a humidified atmosphere for 8 to 13 days and monitored closely for colony growth using an inverted microscope. Within this period, ex vivo tumor growth led to the formation of colonies with a diameter of >50 μm. At the time of maximum colony formation, counts were performed with an automatic image analysis system (Bioreader 5000-Wa Biosys GmbH). 48 hours prior to evaluation, vital colonies were stained with a sterile aqueous solution of 2-(4-iodophenyl)-3-(4-nitrophenyl)-5-phenyltetrazolium chloride (1 mg/ml, 100 μl/well)

Methacycline and paclitaxel inhibited colony formation in a concentration-dependent manner. Methacycline inhibited colony formation of the ovarian cancer cell lines A2780, OVCAR3, OVCAR-5, and SK-OV-3 with IC₅₀ values ranging from 54.11 to 71.07 μM. Bottom plateaus of the concentration-effect curves of responding tumor models ranged between 0% and 56%, indicating that the inhibition of tumor colony growth was not complete in all cases. Based on relative IC₅₀ values, above average activity was observed against 3/8 tumor models (A2780, OVCAR3, and SK-OV-3).

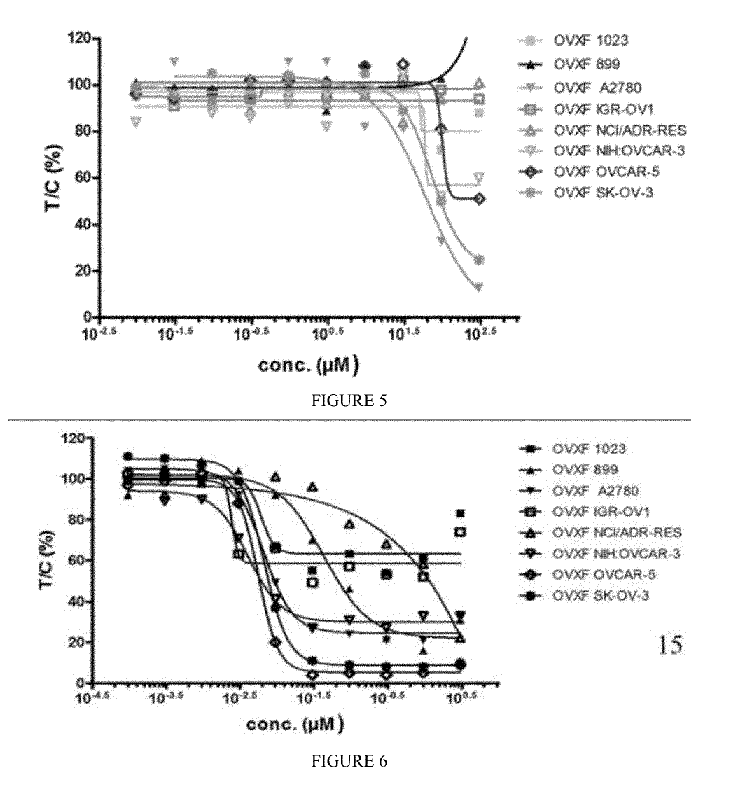

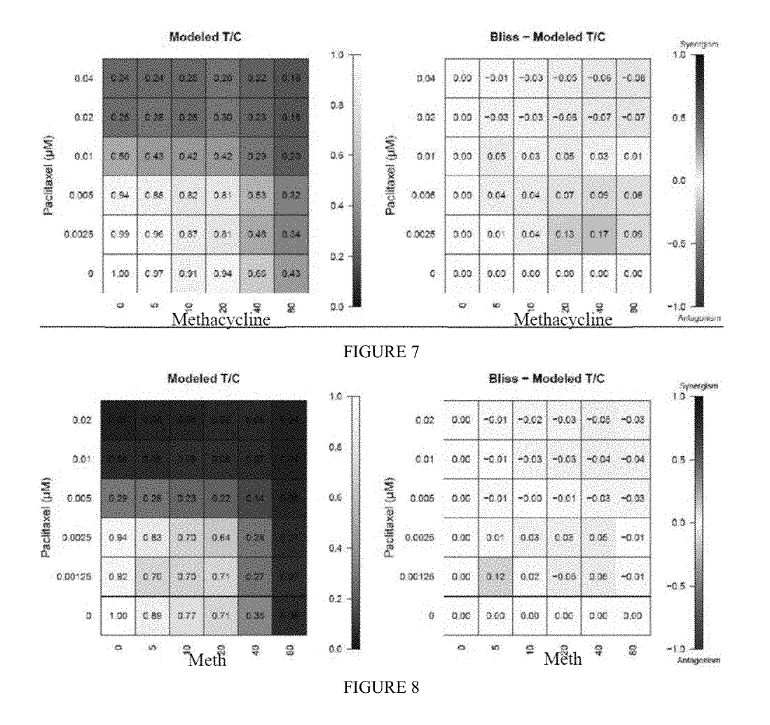

Paclitaxel inhibited colony formation with a mean relative IC₅₀ value of 0.012 μM (mean absolute IC₅₀ value=0.077 Results are depicted in FIGS. 5-6. Bottom plateaus of the concentration-effect curves of the responding tumor models were in the range from 0% to 60%, with a major proportion <35%, indicating clear inhibition of tumor colony growth. Based on relative IC₅₀ values, above average activity was observed against 3/8 tumor models (cell lines IGR-OV1, OVCAR3, and OVCAR-5).

The ability of methacycline and paclitaxel to inhibit ex vivo colony formation of cells with the ability to grow anchorage-independently in semi-solid medium was examined in 8 human tumor cell lines of ovarian cancer. Methacycline inhibited colony formation of the ovarian cancer cell lines A2780, OVCAR3, OVCAR-5, and SK-OV-3 in a concentration-dependent manner. IC₅₀ values were determined ranging from 54.11 to 71.07 μM. Bottom plateaus of the concentration-effect curves of responding tumor models ranged between 0% and 56%, indicating that the inhibition of tumor colony growth was not complete in all cases. Based on relative IC₅₀ values, above average activity (individual IC₅₀ value <½ mean IC₅₀) was observed against 3/8 tumor models (cell lines A2780, OVCAR3, and SK-OV-3) (Table 5). No activity was seen against OVXF 1023L, OVXF 899L, IGR-OV1, and NCI-ADR-RES.

### Example 3: In Vitro Combination Study

The objective of this study was to assess anti-tumor efficacy of methacycline in combination with paclitaxel in a 5×5 matrix combination format against various ovarian cancer cell lines. The compounds were tested in ovarian cell lines, namely OVXF A2780 and OVXF SK-OV-3, obtained from National Cancer Institute (Bethesda, Md., USA). OVXF SKOV-3 cells were obtained from American Type Culture Collection (Rockville, Md., USA). Authenticity of cell lines was confirmed at the DSMZ by STR analysis. Cell lines were routinely passaged once or twice weekly and maintained in culture for up to 20 passages. Cells were grown at 37° C. in a humidified atmosphere with 5% CO₂ in RPMI 1640 medium (25 mM HEPES, with L-glutamine, Biochrom) supplemented with 10% (v/v) fetal calf serum and 0.1 mg/mL gentamicin. The percentage of viable cells was determined in a Neubauer-hemocytometer using trypan blue exclusion.

The CellTiter-Blue® Cell Viability Assay (#G8081, Promega) was used according to manufacturer's instructions. Briefly, cells were harvested from exponential phase cultures, counted and plated in 96-well flat-bottom microtiter plates at a cell density of 4,000-8,000 cells/well depending on the cell line's growth rate. After a 24 h recovery period to allow the cells to resume exponential growth, 10 μL of culture medium (twelve control wells/plate) or of culture medium with test compound were added, and treatment continued for four days. After four days treatment of cells, 20 μl/well CellTiter-Blue® reagent was added. Following an incubation period of up to four hours, fluorescence (FU) was measured by using the Enspire Multimode Plate Reader (excitation λ=531 nm, emission λ=615 nm). For calculations, the mean values of duplicate/6-fold (untreated control) data were used.

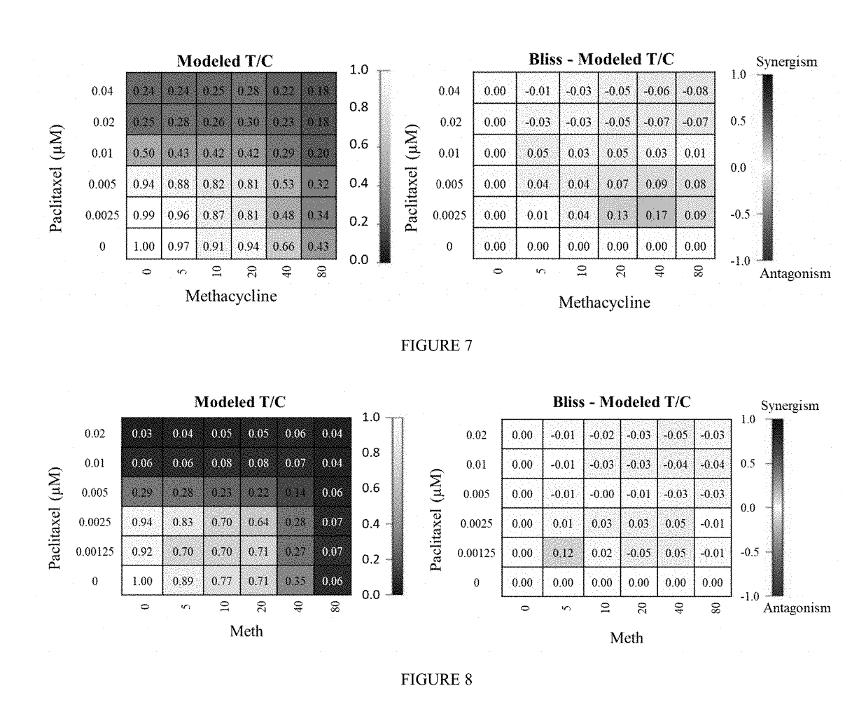

In the present study, methacycline was tested alone and in combination with paclitaxel in order to investigate the ability to inhibit tumor cell growth of ovarian cancer cell lines in a 5×5 matrix combination format. Efficacy of methacycline combined with paclitaxel was assessed by measuring tumor cell viability using a 2D monolayer assay in cell lines OVXF A2780, OVXF IGR-OV1, OVXF SK-OV-3. Results are depicted in FIGS. 7-8. Methacycline tested as single agent inhibited tumor cell viability in a concentration-dependent manner with IC₅₀ values ranging from 34.6 μM (OVXF A2780) to 43.3 μM (OVXF IGR-OV1). Paclitaxel tested as single agent was active with IC₅₀ values ranging from 3 nM (OVXF IGR-OV1) to 9 nM (OVXF SK-OV-3). Additive effects were observed for the combination of methacycline with paclitaxel in ovarian cancer cell lines OVXF SK-OV-3 and OVXF A2780.

Methacycline and paclitaxel inhibited tumor cell viability of OVXF SK-OV-3 cells in a concentration dependent manner. This is also reflected in the matrix combination, where activity of the different combinations was observed with increasing test concentrations of both compounds (FIG. 7, left). Bliss independence analysis showed that overall an additive effect of the combinations was obtained, i.e. neither synergy nor antagonism. No consistent effect was observed across several test conditions. The color coding of the tiles in the heatmap show, that there is no consistent concentration-dependent effect pointing towards synergy (BI>0.15) or antagonism (BI<−0.15). Thus, the oscillation of individual BI values around 0 rather reflects the variability within the assay (FIG. 7, right).

Methacycline and paclitaxel inhibited tumor cell viability of OVXF A2780 cells in a concentration-dependent manner. This is also reflected in the matrix combination, where activity of the different combinations was observed with increasing test concentrations of both compounds (FIG. 8, left). Bliss independence analysis showed that overall an additive effect of the combinations was obtained, i.e. neither synergy nor antagonism. The color coding of the tiles in the heatmap show, that there is no consistent concentration-dependent effect pointing towards synergy (BI>0.15) or antagonism (BI<−0.15). Thus, the oscillation of individual BI values around 0 rather reflects the variability within the assay (FIG. 8, right).

### Example 4: Pharmaceutical Compositions

Tablets

| Ingredients | Quantity mg/tablet |
|---|---|
| Methacycline | 20-300 |
| Microcrystalline cellulose (Avicel PH 102) | 30-150 |
| Silicon dioxide colloidal (Aerosil 200) | 40-160 |
| Sodium starch Glycolate | 30-60 |
| Magnesium stearate | 3-10 |
| Talc | 2-5 |

1. Methacycline, microcrystalline cellulose, colloidal silicon dioxide, sodium starch glycolate were sifted and added to a suitable blender.
2. Magnesium stearate and talc were sifted and added to the blend obtained in of step (1).
3. Blended mixture obtained in step (2) is mixed and compressed into tablets.

Tablets

| Ingredients | Quantity mg/tablet |
|---|---|
| Methacycline | 20-300 |
| Microcrystalline cellulose (Avicel PH 102) | 30-300 |
| Lactose Monohydrate | 15-100 |
| Hydroxypropyl cellulose (Low-substituted) | 10-50 |
| Hydroxypropyl methylcellulose 5 cps | 4-25 |
| Colloidal silicon dioxide | 0.5-5 |
| Magnesium stearate | 3-10 |

1. Methacycline and microcrystalline cellulose was mixed and is then granulated.
2. The wet mass obtained in step (1) was granulation.
3. The obtained granules in step (2) is mixed and blended with hydroxypropyl cellulose, hydroxypropyl methylcellulose, colloidal silicon dioxide, lactose monohydrate, lubricated with magnesium stearate and compressed into tablets.

Capsules

| Ingredients | Quantity mg/tablet |
|---|---|
| Methacycline | 20-300 |
| Pregelatinized corn starch | 10-50 |
| Colloidal silicon dioxide | 1-15 |
| Magnesium stearate | 3-15 |
| Talc | 3-15 |
| Empty hard gelatin capsule | 1-15 |

1. Methacycline, pregelatinized corn starch, Colloidal silicon dioxide and talc were mixed.
2. The blended mixture obtained in step (1) was filled in the empty hard gelatin capsule shells using a capsule filling machine.

Injection

| Ingredients | Quantity mg/mL |
|---|---|
| Methacycline | 20-300 |
| Sodium formaldehyde sulfoxylate | 1.0-2.5 |
| Propyl gallate | 0.1-1.5 |
| Monothioglycerol | 5.0-20.0 |
| Propylene glycol | 0.1-2.0 |
| Monoethanolamine | 0.001-0.2 |
| Magnesium chloride | 5.0-30.0 |
| Citric acid | 2.5-25 |
| Water for Injection | 0.1-1.0 |

1. Water for injection (required quantity) was taken in a suitable vessel, nitrogen gas was bubbled for 20-25 minutes and sodium formaldehyde sulfoxylate was added.
2. Propyl gallate was dissolved in part of propylene glycol and added to the solution of step (1).
3. Monothioglycerol and Methacycline was further added to the solution of step (2) and was mixed.
4. Magnesium chloride was dissolved in part quantity of water for injection and added to the solution of step (2) and the solution was stirred vigorously.
5. pH of the solution was adjusted to ˜8.5 using monoethanolamine.
6. Volume make up was done using propylene glycol, solution was filtered and filled aseptically into type I flint glass vials.

Syrup

| Ingredients | Quantity mg/unit |
|---|---|
| Methacycline | 20-300 |
| Ascorbic acid | 5-15 |
| Sodium hydroxide | 1.5-5.0 |
| Edetate disodium (sodium EDTA) | 0.2-2.0 |
| Saccharin sodium | 0.1-1.0 |
| Sodium metabisulfite (sodium disulfite) | 1-5 |
| Alcohol (ethanol, 95%) | 50-100 |
| Propylene glycol | 75-150 |
| Sorbitol (70% solution) | 75-150 |
| Glycerin (glycerol) | 200-350 |
| Sucrose | 250-400 |
| Quinoline yellow | 0.01-0.08 |
| Pineapple flavor | 0.1-0.5 |
| Purified water | q.s |

1. Sucrose was added to the purified water heated to 90° C. to 95° C. in a suitable vessel and mixed.
2. Propylene glycol, sorbitol (70% solution) glycerin was added to the mixture of step (1) and was allowed to cool with continuous mixing at slow speed.
3. Alcohol was added to the syrup solution of step (2) while mixing at slow speed.
4. Methacycline was added to the solution of step (3) with continuous mixing at high speed until a clear solution was obtained.
5. Ascorbic acid, edetate disodium and sodium metabisulfite were added to the solution of step (4) with continuous mixing at slow speed.
6. Pineapple flavor was dissolved in part quantity of purified water and added to the solution of step (2) with mixing at slow speed.
7. Sodium hydroxide and Saccharin sodium were dissolved in part quantity of purified water and added to the solution of step (2) with slow mixing.
8. Quinoline yellow was dissolved in part quantity of purified water and the colour solution was transferred to the solution of step (2), volume makeup and pH was adjusted with 10% citric acid or 10% sodium citrate solution.
9. The syrup was then filtered and filled in suitable bottles.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method for the treatment of ovarian cancer in a subject, the method comprising administering to said subject methacycline, or a pharmaceutically acceptable salt thereof, in an amount effective to treat ovarian cancer.

2. The method according to claim 1, wherein the ovarian cancer comprises Stage 0, Stage I, Stage II, Stage III or Stage IV ovarian cancer.

3. The method according to claim 1, wherein the ovarian cancer is characterized by the presence of one or more epithelial stromal tumors.

4. The method according to claim 1, wherein methacycline is administered in combination with at least one other cancer therapy.

5. The method according to claim 4, wherein the other cancer therapy comprises surgery, chemotherapy, hormone therapy, immunotherapy or radiation therapy.

6. The method according to claim 5, wherein methacycline is administered in combination with an immunotherapy comprising a monoclonal antibody or an oncolytic virus.

7. The method according to claim 5, wherein the surgery comprises hysterectomy, bilateral salpingo-oophorectomy, debulking, or a combination thereof.

8. The method according to claim 7, wherein methacycline is administered subsequent to surgical treatment for ovarian cancer.

9. The method according to claim 7, wherein methacycline is administered prior to the surgical treatment for ovarian cancer.

10. The method according to claim 7, wherein methacycline is administered prior and subsequent to surgical treatment for ovarian cancer.

11. The method according to claim 5, wherein the chemotherapy comprises administering an anti-cancer agent comprising one or more microtubule inhibitors, topoisomerase inhibitors, platins, alkylating agents, or anti-metabolites.

12. The method according to claim 11, wherein the anti-cancer agent comprises bevacizumab, bleomycin, ifosfamide, etoposide, doxorubicin, pazopanib, cyclophosphamide, doxorubicin, gemcitabine, vinblastine, topotecan, olaparib, carboplatin, cisplatin, paclitaxel, thiotepa, or a combination thereof.

13. The method according to claim 11, wherein platin compound comprises cisplatin, carboplatin, oxaliplatin, or nedaplatin.

14. The method according to claim 13, wherein platin compound is cisplatin.

15. The method according to claim 11, wherein the microtubule inhibitor comprises carbazitaxel, docetaxel, or paclitaxel.

16. The method according to claim 15, wherein the microtubule inhibitor comprises paclitaxel.

17. The method according to claim 5, wherein the hormone therapy comprises tamoxifen.

18. A kit comprising methacycline or a pharmaceutically acceptable salt thereof, and at least one anti-cancer agent comprising one or more monoclonal antibodies, oncolytic viruses, microtubule inhibitors, topoisomerase inhibitors, platins, alkylating agents, or anti-metabolites.

19. A pharmaceutical composition comprising methacycline or a pharmaceutically acceptable salt thereof, at least one anti-cancer agent comprising one or more monoclonal antibodies, oncolytic viruses, microtubule inhibitors, topoisomerase inhibitors, platins, alkylating agents, or anti-metabolites, and a pharmaceutically acceptable carrier.
